(19) United States
(12) Patent Application Publication
(10) Pub. No.: US 2003/0224351 A1
(43) Pub. Date: Dec. 4, 2003

(54) Detection of infectious hematopoietic necrosis virus

(76) Inventors: Kenneth E. Overturf, Buhl, ID (US); Madison S. Powell, Twin Falls, ID (US)

Correspondence Address: HOWARD EISENBERG, 1600 ODS TOWER, 601 S.W. SECOND AVENUE, PORTLAND, OR, 97204-3157, US

(21) Appl. No.: 10/161,588

(22) Filed: Jun. 3, 2002

(51) Int. Cl. C12Q 1/70; C12Q 1/68; C12P 19/34

(52) U.S. Cl. 435/5; 435/6; 435/91.2

(57) ABSTRACT

A real-time PCR method for detecting and quantifying the presence or absence of Infectious Hematopoietic Necrosis Virus (IHNV) in a sample. The method includes the steps of combining with a reverse-transcribed test sample a pair of PCR primers that bind to a cDNA that has been reverse transcribed from a gene of IHNV and a labeled probe that also binds to the cDNA, and performing one or more rounds of PCR on the sample. The probe undergoes a change with each round of amplification of nucleic acid during the PCR reaction, which change causes a change in the signal provided by the label, thus permitting detection of a nucleic acid that is substantially the same as the cDNA, thus providing a detection and, if desired, a quantification, of the level of the IHNV gene in the sample.

Pursuant to 35 U.S.C. §202, it is hereby acknowledged that the U.S. Government has certain rights in the invention described herein, which was supported in part by United States Department of Agriculture-Agriculture Research Service; Project #5366-21310-001-01S.

### FIELD OF THE INVENTION

The invention pertains to the field of detection of the presence of a virus in the environment and in susceptible individuals.

### BACKGROUND OF THE INVENTION

Infectious Hematopoietic Necrosis Virus (IHNV) is a rhabdovirus that causes disease in salmonids, such as salmon and trout species. The IHNV virus is enzootic in the Pacific Northwest portion of the United States as outbreaks of the disease have been reported in Washington, Oregon, and California. The virus has spread beyond the Pacific Northwest and has been reported in other states of the United States, such as Minnesota, Montana, South Dakota, Alaska, and West Virginia, and in Canadian provinces, including British Columbia. The range of the virus now appears to be worldwide as outbreaks have occurred in France, Italy, Belgium, Japan, Taiwan, and Korea.

IHNV infections typically cause severe mortality in young fish, fry, or fingerlings, with reports of up to 80% mortality or severe deformity. Infected fish exhibit externally visible signs of the disease within a week of exposure. Death occurs within four to ten days following exposure, but typically deaths from IHNV cease after about 40 to 50 days.

IHNV, like other rhabdoviruses, is a negative sense RNA virus, the genome of which encodes six genes. The reservoirs of IHNV are clinically infected fish and inapparent carriers among fish. The transmission of IHNV between fish is primarily horizontal, with virus being shed via feces, urine, sexual fluids and external mucus. Cases of vertical transmission, through infected eggs, have also been observed. Once IHNV is established in a population of susceptible fish, the disease is difficult to eliminate because it may become established among carrier fish.

Vaccines are currently under development and testing. However, no vaccine has yet been found to control IHNV infection. Therefore, present control measures for the disease require the identification of infected individuals and measures to prevent uninfected fish from coming into contact with infected individuals and infected environments.

Present methods of testing for the presence of IHNV most commonly occurs by replicating and isolating the virus in established fish cell lines and confirming the identity of the virus through standard serum neutralization assays. This method is sensitive for detecting low concentrations of infectious virus. However, such methods are time and labor intensive and usually provides answers long after the virus has already run its course in a population.

Therefore, other methods that do not require the replication of the virus have been developed. These methods are based upon immunologic methodologies or upon detection of nucleic acids specific for the IHN virus. Included in these methods are in situ hybridization with a probe for the nucleocapsid gene, immunohistochemical and immunogold labeling methods for the detection of virus in vivo, ELISA, and amplification of reverse transcribed (RT) cDNA from tissues using the polymerase chain reaction (PCR).

Routine problems that occur when using standard PCR methods include incorrect interpretation of band presence or absence because of improper primer annealing or buffer concentration. Additionally, the number of cycles to run for correct band amplification can vary between the primers and the type of machine used. These problems are further exacerbated when attempts are made to quantify the level of virus present.

Quantification of IHNV and other viruses is especially important in the management of hatcheries and of protected species that are reared and released back into the wild. In many situations, no harm to fish populations is observed even though a low level of the virus is present in the population. Detection of an increase in the level of virus in the population, however, may indicate the need to alter management procedures to prevent further dissemination and spread of the virus. For endangered fish species, it may be necessary to spawn fish even though virus is detectable in individuals. In this case, those individuals with the lowest levels of virus would be selected for initial spawns. Additionally, in many viral diseases, a low level of virus may not be infectious, but as virus levels increase the possibility of spreading the virus becomes important. Regulations for shipping fish to or from endemic areas often depend on detectable viral levels. Therefore, the ability to quantify viral levels is of critical importance in the management of viral infection.

In recent years, PCR methods have been adapted to provide both for detection and for quantification of nucleic acid sequences in a sample. For example, see, Higuchi, U.S. Pat. No. 6,171,785, incorporated herein by reference. These methods employ forward and reverse primers as in standard PCR plus one or more additional nucleic acid sequences that hybridize to the nucleic acid that is to be amplified. This additional nucleic acid sequence, termed a “probe”, hybridizes to a portion of the nucleic acid to be amplified between the portions that hybridize to the two primers, and is labeled in such a way so that each successive PCR cycle causes a change in the probe or its label. This change in the probe or its label causes activation or accentuation of the label to a degree that is related to the number of additional copies of the amplified nucleic acid during each PCR cycle. By utilizing such methods, referred to as “real-time” PCR, cycle-by-cycle detection of increasing PCR product is achieved by combining thermal cycling with label detection.

Most commonly, the label for the probe is a fluorescent label which provides a fluorescent output signal. This may be achieved by providing a probe which is labeled with a flourescent reporter dye at one end, typically the 5′ end, and a quencher dye at the other, the 3′, end. When the probe is intact, the proximity of the quencher dye to the reporter dye suppresses the fluorescent output of the reporter dye. During each PCR cycle, the 5′ nuclease activity of a DNA polymerase cleaves the probe, which separates the reporter dye from the quencher dye. This separation results in increased fluorescent output of the reporter dye.

During PCR, if the target of interest is present in a sample, the probe will specifically anneal between the forward and reverse PCR primer sites. The nucleolytic activity of the DNA polymerase cleaves the probe between the reporter and the quencher dyes only if the probe hybridizes to the target molecule. The increase in fluorescence is detected only if the target sequence is complementary to the probe and is amplified during PCR. Because of these requirements, non-specific amplification is not detected. Only amplified products that contain the sequence complementary to the probe are recognized by the presence of the fluorescent signal, thereby eliminating certain elements related to the analysis of false-positives. Additionally, one or more other enzymes may be utilized to help limit the amplification of carry over transcription products.

This type of quantitative PCR permits the normalization of pipetting errors and volume changes, which may be done by dividing the reporter fluorescence by a passive reference, contained within each reaction, to determine the normalized reporter signal for each individual reaction. Software may be used to analyze the cycle-to-cycle increase in fluorescence intensity and compare this data to standards in order to determine starting copy numbers for absolute quantification or to compare against other unknown samples for a comparison of relative quantity.

To date, no such method of quantification of IHN virus infection has been reported. The existence of such a method would have a significant impact on the ability to control this disease.

### SUMMARY OF THE INVENTION

In one embodiment, the invention is a method for detection of IHN virus in a sample using real-time PCR. According to this embodiment, a forward primer, a reverse primer, which primers anneal to a DNA sequence that has been reverse transcribed from an RNA gene of the IHN virus, a labeled probe, which probe anneals to the DNA at a site between the sites of annealing of the forward and reverse primers, a DNA polymerase, and the four deoxynucleotide bases A, T, C, and G are combined with a test sample to form a mixture. The reverse transcription of the RNA gene of the IHNV is performed prior to the PCR procedure. The mixture is taken through successive PCR cycles, wherein the probe contains a label that is activated or accentuated to a degree that is related to the number of additional copies of the amplified nucleic acid during each PCR cycle. The PCR cycles include the steps of adjustment to a temperature at which the DNA is separated into single strands, adjustment to a temperature at which the primers and the probe anneal to complementary sequences on the DNA, and adjustment to a temperature at which the polymerase binds and extends a complementary DNA strand from each primer.

In another embodiment, the invention is a primer that binds to a DNA that has been reverse transcribed from an RNA gene of the IHN virus, which primer, when combined with the DNA, a second primer that binds to the DNA, a DNA polymerase, and the four deoxynucleotide bases A, T, C, and G, provides exponential expansion of copies of the DNA during rounds of PCR.

In another embodiment, the invention is a kit that is useful for the detection of IHNV in a sample.

### BRIEF DESCRIPTION OF THE DRAWINGS

FIG. 1 is a graph of the standards for in vitro IHNV nucleocapsid sequence.

FIG. 2 is an amplification plot of the in vitro transcribed nucleocapsid standards and a sample of RNA isolated from the brain of an IHNV infected trout.

FIG. 3 is an amplification plot showing the detection of IHNV isolated from serially diluted water samples.

FIG. 4 is an amplification plot of the sequence detection and identification of IHNV from RNA isolated from the brains and kidneys of infected and control fish.

FIG. 5 is an ethidium bromide staining of serially diluted in vitro transcribed IHNV nucleocapsid gene sequence after standard RT-PCR. Lane 1 contains a 100-bp ladder and lane 12 contains a 1-kb ladder. Lane 10 contains a sample isolated from the brain of an infected animal. Lane 11 contains a positive control of IHNV isolated from tissue culture. The other lanes contain the following dilutions of in vitro transcribed N sequence RNA: Lane 2=0 (negative control); Lane 3=2.4×10⁹; Lane 4=2.4×10⁸; Lane 5=2.4×10⁷; Lane 6=2.4×10⁶; Lane 7=2.4×10⁵; Lane 8=2.4×10⁴; and Lane 9=2.4×10³.

### DETAILED DESCRIPTION OF THE INVENTION

The methods and kits of the present invention are directed to the detection of infectious hematopoietic necrosis virus (IHNV) in a sample, and preferably to the quantification, either absolute or relative, of the virus in the sample. Detection of the virus by the methods and kits is based upon detection of a nucleotide sequence specific to the IHNV, as opposed to presently used methods of viral isolation in cell culture. This permits the detection of the virus in infected fish, as well as in the environment, such as in water, feed, and other reservoirs of infection. The ability to quantify the IHNV provides for the ability to determine changes in the abundance of the IHNV organism over time.

The compositions of the present invention are nucleotide sequences that are primers or probes for the detection of IHNV by PCR, and preferably by real-time PCR, methods. The primers and probes of the invention hybridize to specific portions of a gene of the IHNV. IHNV is a single stranded RNA negative-strand virus, the genome of which possesses 11,131 nucleotides that encode for six genes. The genome is available from GenBank at the National Center for Biotechnology Information (NCBI) of the National Institutes of Health (NIH) and has been assigned Accession no. NC 001652. Preferably, the probes and primers hybridize to portions of either the N or G gene of the IHNV, although probes and primers may hybridize to the other genes of IHNV, such as the P(M1), M2, NV, or L genes.

Methods of PCR and real-time PCR are known in the art. Additionally, other methods of PCR and real-time PCR may be developed in the future and it is intended that such methods be included as suitable for the methods and kits of the invention, so long as such methods are used to detect, preferably quantitatively, the presence or absence of IHNV in a sample.

According to the method of the invention, a pair of DNA sequences, hereafter referred to as “PCR primers” and a third DNA sequence, referred to as a “probe” are exposed to a sample DNA under conditions in which complementary DNA strands will hybridize and the primers and the probe are permitted to either hybridize or not hybridize to two separated portions of the DNA. The primers and the probe bind specifically to a DNA that is reverse transcribed from an RNA gene of IHNV. The primers bind to separated portions of the DNA and the probe binds to a portion of the DNA that is between the portions of the DNA that bind to the primers. The primers, the probe, and the DNA sequence are then exposed to conditions in which the DNA is replicated and then to conditions in which the DNA is denatured. Preferably, multiple cycles of annealing, extension, and denaturation (“PCR cycle”) are performed. By this method, if a DNA that is complementary to the two primers exists in the sample, multiple copies of the DNA are obtained.

The probe is itself a label or contains a label. Each successive PCR cycle causes a change in the probe or its label. This change in the probe or its label causes activation or accentuation of the label to a degree that is related to the number of additional copies of the amplified nucleic acid during each PCR cycle. By utilizing such methods, referred to as “real-time” PCR, cycle-by-cycle detection of increasing PCR product is achieved by combining thermal cycling with label detection.

Preferably, the label is a fluorescent label that increases in intensity to a degree that is related to the number of copies of the DNA that are produced during each PCR cycle. In a preferred embodiment, the probe is a DNA sequence that hybridizes to a portion of a DNA reverse transcribed from a gene of the IHNV at a position between the portions of the DNA where a forward and reverse PCR primer anneal to the DNA, which probe contains a fluorescent reporter dye and a quencher dye, preferably at opposite ends of the probe. The presence of the quencher dye and the reporter dye on the probe causes suppression of the fluorescence caused by the reporter dye. During the extension portion of each PCR cycle, the probe is cleaved, typically by action of a DNA polymerase, which separates the reporter and the quencher dyes, thereby causing an increase in fluorescence which is related to the number of copies of the DNA that are produced during each PCR cycle. The amount of fluorescence is determined after a known number of PCR cycles, which provides a quantitative measure of the number of copies of the DNA that was present in the cycle before PCR.

Because the increase in fluorescence signal is detected only if the target sequence is complementary to the probe, non-specific amplification is not detected by the method of the invention. Only amplified products that contain a sequence that is complementary to the probe are recognized by the giving off of a fluorescent or other signal, false-positives, which are a common problem with standard, non-real time PCR, are eliminated. Software may be used to analyze the cycle-to-cycle increase in fluorescent intensity and to compare this data to standards in order to determine starting copy numbers or to absolutely or relatively quantify the viral sequence present in the sample.

The invention is illustrated below with reference to particular genes of IHNV and to particular DNA sequences, including particular primers and probes. One skilled in the art will understand that the invention is applicable to genes of IHNV other than those exemplified below and that primers and probes other than those disclosed herein, the DNA sequence of which other primers and probes may be determined by methods disclosed herein, may be used in accordance with the invention. One skilled in the art will further understand that, because the genes of IHNV are RNA sequences, the genes must be reverse transcribed to obtain a DNA sequence before or during the PCR procedure.

In a preferred embodiment of the method of the invention, the gene of IHNV that is amplified is the N (nucleocapsid) gene. In an alternative preferred embodiment, the gene is the G (glycoprotein) gene. In less preferred embodiments, the gene is either the M1, M2, NV, or L gene. In accordance with each of these embodiments, the gene or a portion thereof is amplified by polymerase chain reaction, preferably by real-time polymerase chain reaction to provide quantification of the number of copies of the gene that is present in a sample.

When performing the method of the invention to determine the presence in the sample of the N gene of IHNV, a preferred forward primer has the sequence:

5′-CAAGCGGGCGGTGAGTC-3′, Seq. ID No. 1,

a preferred reverse primer has the sequence:

5′-GTTCGGCGACCGTACCTG-3′, Seq. ID No. 2.,

and a preferred probe has the sequence:

5′-CATTGAGCCTCCTGTGCGCGTTC-3′, Seq. ID No. 3.

When performing the method of the invention to determine the presence in the sample of the G gene of IHNV, a preferred forward primer has the sequence:

5′-CATACCTCCTATCCAAATTCCGATC-3′, Seq. ID No. 4,

a preferred reverse primer has the sequence:

5′-TCCACAGCGACCGTCATG-3′, Seq. ID No. 5,

and a preferred probe has the sequence:

5′-CCCGGAATAAATGACGTCTACGCTATGCAC-3′, Seq. ID No. 6.

The reverse primers of Seq. ID Nos. 2 and 5 were designed to prime both the reverse transcription reaction of the N and G gene, respectively, and the amplification of cDNA obtained by reverse transcription of these genes. This is preferred, but is not essential for the invention. Other means of obtaining both amplification and reverse transcription, many of which are known in the art, are suitable for the method of the invention. For example, an RNA gene of IHNV may be reversed transcribed in a separate reaction to provide a cDNA and such cDNA may be utilized in the method of the invention. Additionally, primers and probes other than those disclosed above are suitable for the method of the invention. Such other primers and probes may be obtained, for example, by the following methods.

To determine a suitable forward primer having a sequence other than Seq. ID No. 1, a candidate DNA sequence is determined that will hybridize to a portion of the N gene of IHNV that is 5′ to the positions where the primer of Seq. ID No. 2 and the probe of Seq. ID No. 3 anneal. One or more PCR cycles of annealing, extension, and denaturation are performed with a sample containing a known quantity of the N gene, the primer of Seq. ID No. 2, and the probe of Seq. ID. No. 3. Following PCR, the quantity of DNA that has been amplified is determined. The candidate forward primer is suitable for the method of the invention if it, together with the other necessary components for PCR, produced an exponential expansion of copy number of the DNA during each round of PCR. In this way, the suitability of any candidate forward primer may be determined.

If desired, such a PCR protocol may be performed in parallel to a PCR protocol using the primers and probe of Seq. ID Nos. 1 to 3, whereby the number of copies obtained by a given number of PCR rounds with a candidate forward primer is compared with that obtained by the same number of PCR rounds using the exemplified forward primer of Seq. ID No. 1.

Suitable reverse primers may be determined in a similar manner, but utilizing a candidate reverse primer, the primer of Seq. ID No. 1 as a forward primer, and the probe of Seq. ID No. 3. Likewise, suitable probes may be determined by utilizing the primers of Seq. ID Nos. 1 and 2 and a candidate probe.

Suitable primers and probes for the methods of the invention to determine the presence of the G gene of IHNV may be obtained by the method described above for obtaining suitable primers and probes to determine the presence of the N gene. In this case, the forward primer of Seq. ID No. 4 replaces that of Seq. ID NO. 1, the reverse primer of Seq. ID No. 5 replaces that of Seq. ID NO. 2, the probe of Seq. ID No. 6 replaces that of Seq. ID No. 3, and the G gene replaces the N gene.

When performing the method of the invention so as to obtain a quantitative assessment of the amount of IHNV, or a gene of IHNV, in a sample, the amplification of a test sample may be compared to the amplification of a control standard or series of standards to determine an absolute quantity of IHNV in the test sample. Alternatively, the amplification of a test sample may be compared to the amplification of a second sample in order to determine a relative quantity of IHNV in the two samples. The ability to quantify IHNV virus, either absolutely or relatively, is an improvement over standard PCR methods for sequence quantification. In the real-time method according to the invention, the relative measurements in the sequence detector occur during the log phase of amplification. In contrast, with standard PCR, such measurements occur at the end of the reaction when amplification has reached a plateau stage.

Because IHNV possesses a single-stranded RNA genome, the presence of transcriptional and genomic material cannot be distinguished. Therefore, for quantification purposes, it is preferred to standardize the levels of virus over a range that avoids both extremely high levels in which the transcriptional material would overwhelm the genomic number and very low levels where a few transcriptionally active organisms could provide a false reading of a high initial copy number. Because a sample may contain very low copy numbers, it is preferred to use the N or the G genes of IHNV. Neither of these genes are involved in cell control, but rather they code for portions of the virus itself. Therefore, the potential problem of overcounting because of high transcription of cell control genes is avoided using the N or G genes.

The present invention provides for one or more kits containing the elements required to detect the presence of IHNV in a sample using real-time PCR. The kit includes a container housing a forward primer which is a DNA sequence that binds, as described above, to a nucleotide sequence that has a sequence of a DNA that is reversed transcribed from a gene of IHNV. The kit further includes a container housing a reverse primer which is a DNA sequence that binds, as described above, to a nucleotide sequence that is has a sequence of a DNA that is reversed transcribed from the IHNV gene, the binding by the reverse primer being at a site on the DNA that is distinct from the site at which the forward primer binds. The kit further contains a probe which is a nucleotide sequence that binds to a site on the DNA between the sites of binding of the reverse and forward primers, wherein the probe is labeled in such a way that the label is activated or accentuated during each cycle of PCR in an amount that is related to the number of additional copies of the DNA that are amplified during the cycle. The primers and probes described above may be housed in the same or in separate containers. The kit optionally contains additional components for carrying out PCR, such as dNTPs, a DNA polymerase, and a reverse polymerase, and instructions for performing PCR and/or real-time PCR.

The DNA sequence preferably is a cDNA of a gene of IHNV, most preferably the N or G genes. The forward primer preferably has the sequence of Seq. ID No. 1, which binds to the N gene, or of Seq. ID No. 4, which binds to the G gene. The reverse primer preferably has the sequence of Seq. ID No. 2, which binds to the N gene, or of Seq. ID No. 5, which binds to the G gene. The probe preferably has the sequence of Seq. ID No. 3, which binds to the N gene, or of Seq. ID No. 6, which binds to the G gene. However, any primer or probe that is suitable for the method of the invention, as described above, is suitable for the kit of the invention.

Preferably, the probe is labeled with a fluorescent label, the intensity of which increases to a degree related to the number of additional copies of the DNA that are made with each cycle of PCR. However, labels other than fluorescent labels are suitable for the kit and method of the invention, so long as the intensity of the label increases to a degree that is related to the number of additional copies of the DNA that are obtained with each cycle of PCR.

Optionally, the kit may contain one or more enzymes that are used in the real-time PCR method of the invention. The kit may also contain any additional materials needed to carry out the method of the invention, such as buffers, pipettes, nucleic acids, tubes, and the like.

The invention is illustrated by the following non-limiting examples.

### EXAMPLE 1

Virus and Cell Culture

An epithelioma papulosum cyprini (EPC) cell line (Fijan et al., Annals of Virology, 134:207-220 (1983)), and chinook salmon embryo (CHSE-214) cell line (Lannan et al., In Vitro, 20:671-676 (1984)), were used for the propagation and isolation of IHNV. Cells were maintained at 15-25° C. in minimum essential medium (MEM) (GIBCO, Lifetechnologies, Grand Island, N.Y., USA) supplemented with 10% fetal bovine serum (Hyclone, Logan, Utah, USA) and 2 mM L-glutamine (GIBCO, Lifetechnologies). The IHNV isolate 220-90 (LaPatra et al., Diseases of Aquatic Organisms, 20:119-126 (1994)) was used to produce stock virus. Virus used for fish injections was also identified and quantified by plaque assay procedures (LaPatra et al., Journal of Aquatic Animal Health, 1:29-36 (1989)) using the EPC and SSN-1 (striped snakehead fish, European Collection of Cell Cultures No. ECACC 96082808) cell lines (LaPatra et al., American Fisheries Society Fish Health Section Newsletter, 27:3-4 (1999)).

### EXAMPLE 2

In Vitro Transcription of Control mRNAs

Separate plasmids containing either the N or G gene from the Western Regional Aquaculture Consortium (WRAC) IHNV isolate accession no. L40883 (Morzunov et al., Virus Research, 38:175-192 (1995)) were used. These plasmids contain either the N or G gene RT-PCR amplified and blunt-end cloned into the EcoRV site of the vector pT7Blue (Novagen, Madison, Wis., USA). The plasmids were termed WRAC-N and WRAC-G, respectively.

These vectors were linearized with the enzyme BAmHI, and then gel purified. The RNA transcripts were produced using the RiboProbe In Vitro Transcription System (Promega, Madison, Wis., USA) by priming transcription off the pT7Blue T7 polymerase priming site. Transcripts were run on formaldehyde/MOPS gels to ensure the presence of a single band corresponding to the correct size. Quantification of isolated transcripts was performed by spectrophotometer and by fluorometer with RiboGreen (Molecular Probes, Eugene, Oreg., USA). The transcripts were then used as quantitative standards in the analysis of test samples.

### EXAMPLE 3

RNA Isolation from Tissues

Specific pathogen-free rainbow trout (Oncorhynchus mykiss) were experimentally infected by intraperitoneal injection with 100,000 plaque forming units (PFU) of IHNV. The trout were killed and, immediately after killing, tissues isolated from the brain and kidney of the infected fish and of control (sham infected) fish were quick-frozen in liquid nitrogen in 2 mL cryo-vials. The tissues were maintained at −80° C. until the time of RNA isolation. Within one week after kill, the tissue samples were thawed and total RNA was isolated using the TRIZol extraction method (GIBCO, Lifetechnologies). The quantity and purity of the RNA was determined by analysis on a spectrophotometer at 260 nM.

### EXAMPLE 4

Standard Reverse Transcription PCR (RT-PCR)

Primers were generated for reverse transcription and amplification of regions within the N (nucleotides 175-1350) and G (nucleotides 2999-4575) genes. Reverse transcription and amplification protocol was by addition of RNA to a master mix solution as per the Reverse-iT one step protocol (Advanced Biotechnologies Lted., Surrey, UK). The PCR cycle reactions were as follows - 30 minutes at 47° C., 2 minutes at 94° C., followed by 35 or 40 cycles of 20 seconds at 94° C., 30 seconds at 55° C., and 1 minute at 72° C. A fixed extension time of 5 minutes at 72° C. was added on after the amplification cycles. After the PCR run, the samples were run on a 1.5% agarose (BioWhittaker Molecular Applications, Rockland, Me., USA) gel, ethidium stained and visualized using a BioDoc-IT 2UV Gel Imaging Workstation (UVP, Upland, Calif., USA).

### EXAMPLE 5

Sequence Detection

After quantification, 100 ng of total isolated RNA from each sample was added to a microcentrifuge tube containing 1× TaqMan buffer (Perkin Elmer/Applied Biosystems, Foster City, Calif., USA), 3 mM MnOAc, 0.3 mM dNTPs except for dTTP, 0.6 mM dUTP, 0.3 μM forward primer, 0.3 μM reverse primer, 0.2 μM FAM-6 (6-carboxyfluorescein) labeled probe, 5 units rTH DNA polymerase (Perkin Elmer/Applied Biosystems), and 0.5 units AmpErase UNG enzyme (Perkin Elmer/Applied Biosystems). The primers and fluorescent labeled probes used for the N and G genes, respectively, had the sequences of Seq. ID Nos. 1 to 3 and 4 to 6. The probes were labeled with the fluorescent tag 6-FAM.

Fifty microliters from each sample were then pipetted into individual wells of a 96-well optical plate, capped, and placed into an ABI-Prism model 7700 sequence detector (PE Applied Biosystems, Foster City, Calif., USA), an instrument that monitors the accumulation of PCR products by measuring the fluorescent output signal from a probe-specific primer. Reverse transcription and polymerase chain reaction conditions were as follows - 2 minutes at 50° C., 30 minutes at 60° C., 5 minutes at 90° C., and then 40 cycles of PCR of 20 seconds at 92° C. followed by 1 minute at 62° C.

The fluorescence output for each cycle was measured and downloaded to a Macintosh G3 computer upon the completion of the entire run. Accumulated data was analyzed using the computer program Sequence Detector version 1.7 (Applied Biosystems, Foster City, Calif., USA).

### EXAMPLE 6

Standardization of Samples

Clones for the IHNV genes G and N were obtained and used for the preparation of in vitro transcribed MRNA. The RNA was then quantified and the molecular weights for each transcript were determined. The lengths and weights for the transcripts of the IHNV genes were such that for N, which is 1175 nucleotides in length, the molecular weight was calculated to be 379,416 g mol⁻¹, and for G, which is 1526 nucleotides in length, the molecular weight was calculated to be 488,698 g mol⁻¹. These figures were then used to calculate the number of transcripts per microgram of starting material. Copy numbers for each sequence per unit volume were 2.4×10¹² copies per μL for N and 1.6×10¹² copies per μL for G.

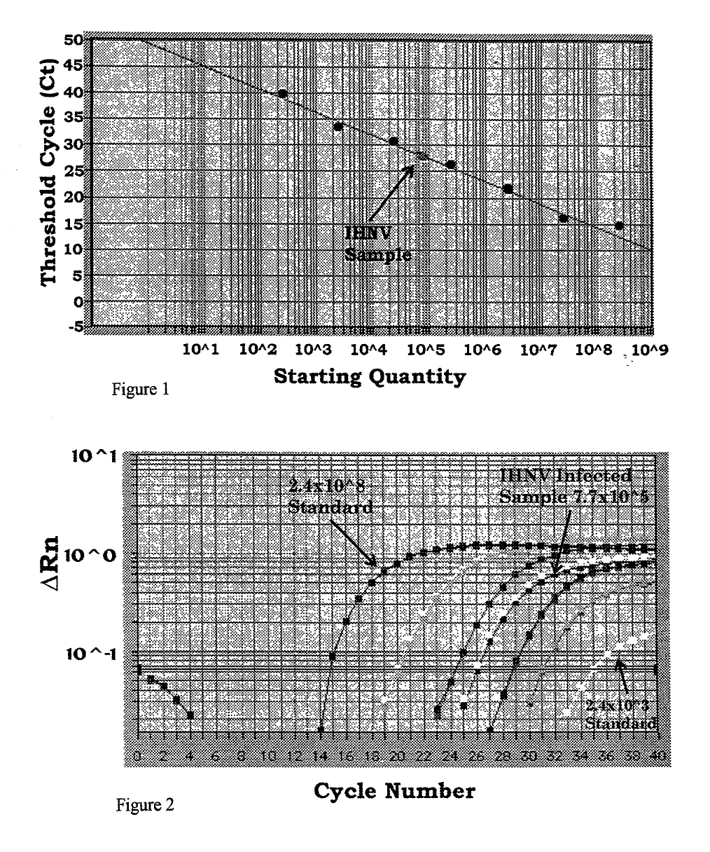

Dilutions corresponding to 1:10 were made until the final concentrations had been diluted to less than 100 copies per μL, an 11-fold dilution. After running the reactions, the results were plotted as the threshold cycle (Ct), which corresponds to the cycle at which a statistically significant increase in fluorescent output occurs, against the starting quantity number. As shown in FIG. 1, the N in vitro transcript gave a linear regression. The slope of regression was −4.383 and the correlation coefficient was 0.9985. The graph of FIG. 1 demonstrates that the higher the initial copy number in the sample, the earlier the PCR goes into a log phase of amplification and that the initial copy number and the threshold cycle are directly correlated. This permits the accurate quantification of an unknown sample.

RNA isolated from a known infected IHNV control was also run to demonstrate the accuracy level of detection. The change in fluorescent signal (ΔRN), normalized to a passive reference signal, for each cycle of amplification is shown in FIG. 2. As shown, up to a certain level, increasing concentrations of a standard is represented by the measured accumulation of transcript and fluorescence output going into log phase and before the occurrence of sequence plateau levels after several cycles.

The results for in vitro transcribed G transcript mimicked those of the N transcript and are not shown.

Using these standards, detected fluorescence signals from unknown samples may be compared and initial concentrations of the unknown transcripts can be determined.

### EXAMPLE 7

Detection of IHNV in Water Samples

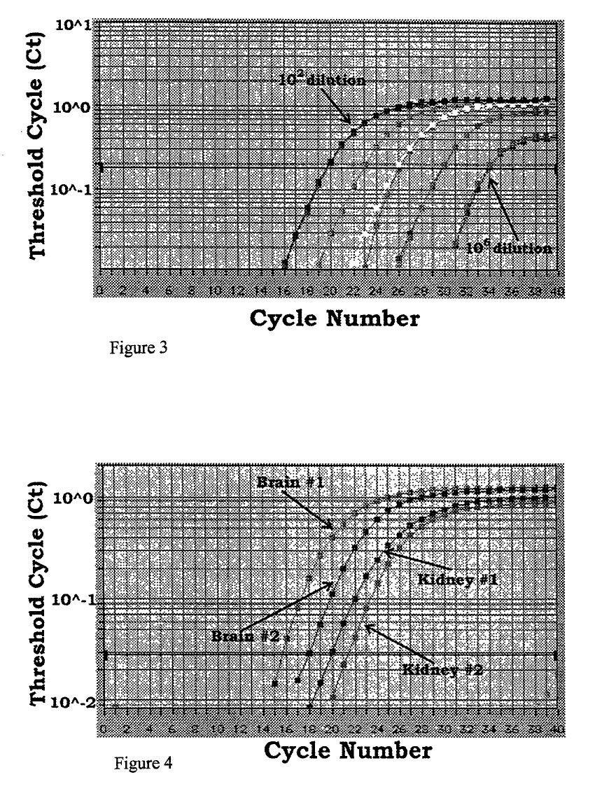

The IHNV isolate 220-90 (LaPatra et al., Diseases of Aquatic Organisms, 20:119-126 (1994)), was used to produce stock virus. The stock virus was serially diluted 10⁻¹ to 10⁻¹⁰ and aliquoted into vials. The initial concentration of virus, as determined by plaque assay, was 2.2×10⁸ PFU mL⁻¹ in SSN-1 cells. From each dilution, virus was isolated from a 250 μL aliquot, and resuspended in 30 μL of Tris-EDTA (TE). Virus was readily detectable by the method of the invention from samples diluted 1×10⁶ fold, or at the level of less than 1000 virions per 250 μL sample. The data is shown in FIG. 3, which is a graph of the change in fluorescent signal compared to a passive reference signal (ΔR) against number of PCR amplification cycles.

### EXAMPLE 8

Detection of IHNV from Isolated Tissues

Kidney and brain homogenates were removed from infected fish and then subsequently processed for sequence identification. Samples from infected individuals were run alongside non-infected controls, and in no case was there evidence of false-positive detection. Samples were also tested by plaque assay to determine presence of infection. The presence of virus was detected in samples from all infected individuals in both brain and kidney.

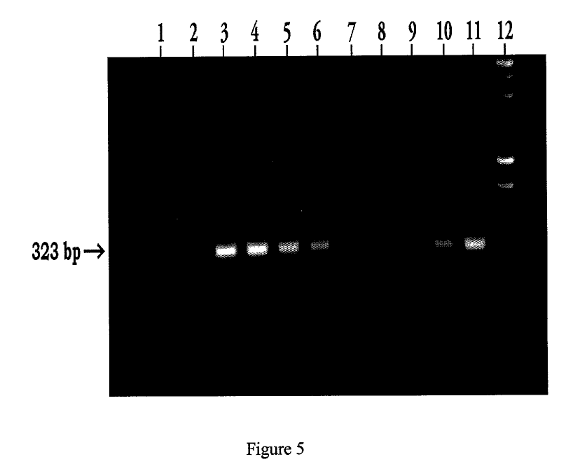

RNA was isolated from the brain and kidney of two infected and one control fish and the RNA was tested according to the method of the invention. The data is shown in FIG. 4, which is a graph of the measurement of the change in fluorescent signal compared to a passive reference signal (ΔR) plotted against cycle number of PCR. As shown in this figure, IHNV is readily detectable in the tissues of infected fish while the control fish is completely negative. A spurious amplification of a negative control is shown in the lower right hand comer of FIG. 4, but this amplification was not detectable at the logarithm measurement line.

### EXAMPLE 9

Comparison of Sequence Detection Method of the Invention to Standard RT-PCR Quantification

A panel of in vitro transcribed N mRNA was prepared containing dilutions ranging from 10³ to 10⁹. The initial substrate concentration was 1.48 μg/μL or 2.4×10¹² copies per μL. The samples were prepared side by side for both standard RT-PCR (reverse transcription PCR) and sequence detection, and 5 μL of diluted material from each dilution was run in reactions in an ABI 7700 sequence detector and in a Perkins Elmer 2400 PCR machine (Perkin Elmer, Inc., Wellesley, Mass., USA) (50 μL/reaction). Two different RT-PCR procedures were performed with either 35 or 40 rounds of amplifications.

It was observed that increasing the number of PCR amplification rounds in standard RT-PCR did not increase the sensitivity of the method. The standard RT-PCR run sample diluents were run on a 1.5% agarose gel and relative quantification and detection comparisons were made between this standard RT-PCR and the method of the invention.

The standard RT-PCR was only able to reliably detect the presence of the N in vitro transcribed RNA to a level of 1×106 copies, as visualized by ethidium bromide staining, shown in FIG. 5. The level of sensitivity using the method of the invention was identical to that shown in FIG. 2, as in vitro transcribed RNA was detectable in the range of 1×10³ copies.

The invention provides methods, kits, and nucleic acid sequences that are useful for monitoring and evaluating the presence of IHNV in the environment and within a population and for appraising the carrier status of infected individuals. The speed and accuracy of the method make it especially useful for the detection of pathogenic organisms from tissue or water samples.

In evaluating quantitative measurements of organism presence, the method of the invention is superior to known methods because, according to the invention, detection of the IHNV organism is directly measured during the DNA amplification stage of PCR via a signal from a labeled probe which probe specifically identifies a sequence unique to the IHNV organism. An advantage of the method of the invention over standard PCR detection methods is that no further steps are required for detection of minute quantities of IHNV.

The use of a specifically labeled probe is another advantage as this helps to eliminate a problem associated with classic PCR methods of mispriming. In PCR protocols other than in accordance with the invention, PCR products are analyzed by the presence or absence of bands, and the presence of false priming sequences can lead to the amplification of non-target DNA. This problem is overcome according to the invention as a signal from the probe label is relayed only upon the amplification of a nucleic acid sequence that specifically hybridizes to the probe, and preferably upon cleavage of the probe upon such amplification.

The invention also provides a vast improvement in the determination of absolute quantities of IHNV organisms. This is achieved by comparing the amplification and label output of an unknown sample at each PCR cycle to a series of standards and passive control sample signals that are preferably run concurrently with the unknown sample.

The improvement in quantification is also obtained in determining relative quantities of IHNV. In this case, experimental samples are compared with each other and with a passive reference at each cycle rather than to a true standard.

The improvement in quantification by the method of the invention compared to standard, non-real-time, PCR is at least partially due to the fact that the absolute and relative measurements obtained according to the method of the invention are obtained during the log phase of amplification and not at the end of the reaction as with standard PCR, when amplification of sequences has reached a plateau state. Additionally, standard PCR may provide false information of quantity because, for example, if the number of cycles performed is too high, this may falsely elevate low level numbers of sequence, making it appear as if there was substantially more starting material in samples containing low numbers of sequence copies.

The method of the invention also has advantages over present quantification methods such as plaque analysis methods. During plaque analysis methods quantities of a sample are typically filtered to obtain a sample that may be more readily prepared and to concentrate the numbers of pathogens in the sample. During the concentration procedure, a large portion of the infective virus organisms may die or become unable to generate an active infection. Such organisms are then unable to be detected by plaque analysis. However, because the method of the invention does not require the use of live or infectious organisms, the method of the invention may provide a more accurate assessment of quantity of organisms in a sample. A major improvement provided by the method of the invention over plaque assay analysis, which is the current primary method of analysis for IHNV, is the speed of real-time PCR analysis that can be performed within a day. In contrast, plaque analysis typically takes up to two weeks before viral plaques plated in tissue culture are able to be seen and counted.

All articles and patents cited in this application are incorporated herein by reference.

Further modifications, uses, and applications of the invention described herein will be apparent to those skilled in the art. It is intended that such modifications be encompassed in the following claims.

1. A method for detecting the presence or absence of Infectious Hematopoietic Necrosis Virus (IHNV) in a sample by real-time polymerase chain reaction (PCR) comprising:
(a) combining the sample, a forward PCR primer that hybridizes to a portion of a cDNA that has been reverse transcribed from a gene of IHNV, a reverse PCR primer that hybridizes to a second portion of the cDNA which is distinct from the portion of the cDNA to which the forward primer hybridizes, and a labeled probe that hybridizes to a portion of the cDNA that is between the portions of the cDNA to which the forward and reverse primers hybridize and which label provides a change in signal that is related to the number of copies of a DNA that has a sequence substantially identical to the cDNA that are amplified during each round of PCR,
(b) performing one or more rounds of PCR on the sample,
(c) and determining the amount of change in the signal of the label, thereby detecting the presence or absence of IHNV in the sample.

2. The method of claim 1 wherein the sample is exposed to a reverse transcriptase before the performing of the PCR.

3. The method of claim 1 wherein the IHNV gene is the N gene.

4. The method of claim 3 wherein the forward PCR primer comprises the sequence of Seq. ID No. 1.

5. The method of claim 3 wherein the reverse PCR primer comprises the sequence of Seq. ID No. 2.

6. The method of claim 3 wherein the probe comprises the sequence of Seq. ID No. 3.

7. The method of claim 1 wherein the IHNV gene is the G gene.

8. The method of claim 7 wherein the forward PCR primer comprises the sequence of Seq. ID No. 4.

9. The method of claim 7 wherein the reverse PCR primer comprises the sequence of Seq. ID No. 5.

10. The method of claim 7 wherein the probe comprises the sequence of Seq. ID No. 6.

11. The method of claim 1 wherein the label is a fluorescent label.

12. The method of claim 1 which further comprises calculating the quantity of IHNV in the sample by determining the amount of change in the signal of the label following a known number of rounds of PCR.

13. A deoxyribonucleotide sequence that hybridizes to a cDNA that is reverse transcribed from a gene of Infectious Hematopoietic Necrosis Virus (IHNV), wherein the sequence, when combined with the cDNA and a second deoxyribonucleotide sequence that hybridizes to a portion of the cDNA that is distinct from the portion of the cDNA to which the first deoxyribonucleotide sequence hybridizes, provides an exponential amplification of the cDNA when the cDNA is amplified by polymerase chain reaction (PCR).

14. The deoxyribonucleotide sequence of claim 13 wherein the gene of IHNV is the N gene.

15. The deoxyribonucleotide sequence of claim 14 which comprises the sequence of Seq. ID No. 1.

16. The deoxyribonucleotide sequence of claim 14 which comprises the sequence of Seq. ID No. 2.

17. The deoxyribonucleotide sequence of claim 13 wherein the gene of IHNV is the G gene.

18. The deoxyribonucleotide sequence of claim 14 which comprises the sequence of Seq. ID No. 4.

19. The deoxyribonucleotide sequence of claim 14 which comprises the sequence of Seq. ID No. 5.

20. A deoxyribonucleotide sequence that hybridizes to a cDNA that is reverse transcribed from a gene of Infectious Hematopoietic Necrosis Virus (IHNV), wherein the sequence, when combined with two PCR primers that bind to the cDNA, produces a signal that is produced or that is increased in relation to the number of copies of the cDNA that are produced during PCR.

21. The deoxyribonucleotide sequence of claim 20 which binds to a portion of the cDNA that is between the sites on the cDNA at which the two PCR primers bind.

22. The deoxyribonucleotide sequence of claim 20 wherein the gene of IHNV is the N gene.

23. The deoxyribonucleotide sequence of claim 22 which comprises the sequence of Seq. ID No. 3.

24. The deoxyribonucleotide sequence of claim 20 wherein the gene of IHNV is the G gene.

25. The deoxyribonucleotide sequence of claim 24 which comprises the sequence of Seq. ID No. 6.

26. The deoxyribonucleotide sequence of claim 20 wherein the signal is a fluorescent signal.

27. A kit for detecting by real-time PCR the presence of IHNV in a sample, comprising a forward PCR primer that hybridizes to a portion of a cDNA that has been reverse transcribed from a gene of IHNV, a reverse PCR primer that hybridizes to a second portion of the cDNA which is distinct from the portion of the cDNA to which the forward primer hybridizes, and a labeled probe that hybridizes to a portion of the CDNA that is between the portions of the cDNA to which the forward and reverse primers hybridize and which label provides a change in signal that is related to the number of copies of a DNA that has a sequence substantially identical to the cDNA that are amplified during each round of PCR.

28. The kit of claim 27 which further comprises a reverse transcriptase.

29. The kit of claim 27 wherein the gene of IHNV is the N gene.

30. The kit of claim 29 wherein the forward PCR primer comprises the sequence of Seq. ID No. 1.

31. The kit of claim 29 wherein the reverse PCR primer comprises the sequence of Seq. ID No. 2.

32. The kit of claim 29 wherein the probe comprises the sequence of Seq. ID No. 3.

33. The kit of claim 27 wherein the gene of IHNV is the G gene.

34. The kit of claim 33 wherein the forward PCR primer comprises the sequence of Seq. ID No. 4.

35. The kit of claim 33 wherein the reverse PCR primer comprises the sequence of Seq. ID No. 5.

36. The kit of claim 33 wherein the probe comprises the sequence of Seq. ID No. 6.

37. The kit of claim 27 wherein the label is a fluorescent label.


SEQUENCE LISTING (6 sequences)

SEQ ID NO: 1 - DNA, 17 bp, Artificial Sequence
  Primer that binds to a cDNA reverse transcribed from the N gene of Infectious Hematopoietic Necrosis Virus, which has been assigned GenBank Accession No. NC 001652
caagcgggcggtgagtc

SEQ ID NO: 2 - DNA, 18 bp, Artificial Sequence
  Primer that binds to a cDNA reverse transcribed from the N gene of Infectious Hematopoietic Necrosis Virus, which has been assigned GenBank Accession No. NC 001652
gttcggcgaccgtacctg

SEQ ID NO: 3 - DNA, 23 bp, Artificial Sequence
  Probe that binds to a cDNA reverse transcribed from the N gene of Infectious Hematopoietic Necrosis Virus, which has been assigned GenBank Accession No. NC 001652
cattgagcctcctgtgcgcgttc

SEQ ID NO: 4 - DNA, 25 bp, Artificial Sequence
  Primer that binds to a cDNA reverse transcribed from the G gene of Infectious Hematopoietic Necrosis Virus, which has been assigned GenBank Accession No. NC 001652
catacctcctatccaaattccgatc

SEQ ID NO: 5 - DNA, 18 bp, Artificial Sequence
  Primer that binds to a cDNA reverse transcribed from the G gene of Infectious Hematopoietic Necrosis Virus, which has been assigned GenBank Accession No. NC 001652
tccacagcgaccgtcatg

SEQ ID NO: 6 - DNA, 30 bp, Artificial Sequence
  Probe that binds to a cDNA reverse transcribed from the G gene of Infectious Hematopoietic Necrosis Virus, which has been assigned GenBank Accession No. NC 001652
cccggaataaatgacgtctacgctatgcac